(19) United States
(12) Patent Application Publication
(10) Pub. No.: US 2009/0043129 A1
(43) Pub. Date: Feb. 12, 2009

(54) POTENT AND SELECTIVE LIGANDS OF CANNABINOID RECEPTORS

(76) Inventors: Antonella Brizzi, Siena (IT); Vittorio Brizzi, Siena (IT); Rossella Sirianni, Siena (IT); Vincenzo Di Marzo, Siena (IT); Maria Grazia, Siena (IT); Tiziana Bisogno, Siena (IT)

Correspondence Address: JUNEAU PARTNERS, P.O. BOX 2516, ALEXANDRIA, VA, 22301, US

(21) Appl. No.: 11/814,483

(22) PCT Filed: Jan. 24, 2006
(86) PCT No.: PCT/IT2006/000039

§ 371 Date: Apr. 6, 2008

(30) Foreign Application Priority Data
Jan. 25, 2005 (IT) RM2005A000037
Aug. 9, 2005 (IT) RM2005A000438

(51) Int. Cl. C07C 233/11; A61K 31/165; A61P 29/00; A61P 3/00; A61P 35/00

(52) U.S. Cl. 564/175; 514/622

(57) ABSTRACT

The present invention relates to high affinity compounds, able to bind CB₁ and CB₂ endocannabinoid receptors. The compounds of the invention find particular application as agents for pain therapy and/or anti-inflammatory and/or anti-stress and/or anti-oxidising and/or hypotensive and/or immune suppressive therapy and/or anti-spastic activity in multiple sclerosis and/or anti-cancer.

### TECHNICAL FIELD OF THE INVENTION

The present invention relates to high affinity compounds, able to bind at least one of the receptors of the endocannabinoid system, CB₁ and CB₂. The compounds of the invention find particular application in all medical applications involving said receptors, in particular as agents for pain therapy and/or anti-inflammatory and/or anti-stress and/or anti-oxidising and/or hypotensive and/or immune suppressive therapy and/or anti-spastic activity in multiple sclerosis and/or anti-cancer.

### PRIOR ART

The soothing, analgesic and anti-convulsive properties, as well as the ability to induce mental and judgement distortions, of marijuana and hashish, both derivatives of Cannabis sativa L., and their derivatives, mainly of Δ⁹-THC o(−)-(trans)-Δ⁹-tetrahydrocannabinol (THC), have been known for years. Cannabis sativa contains about sixty cannabinoids, responsible for the pharmacological effects. During the past two decades, the first proteic receptor, called CB₁, of the endocamiabinoid system at the central nervous system level was identified (Devane et al. 1988). Moreover, the first endogenous agonist, Anandamide or arachidonylethanolamide or cis-5,8,11,14-eicosatetraenoylethanolamide (AEA, Devane et al. 1992), was isolated and subsequently the second proteic receptor, called CB₂ (Munro et al. 1993), present on the cells of the immune system of mammals, but not at the level of the central nervous system. Lastly, a second endogenous agonist, with non amidic structure, identified as 2-arachidonylglycerol or 2-AG, was discovered.

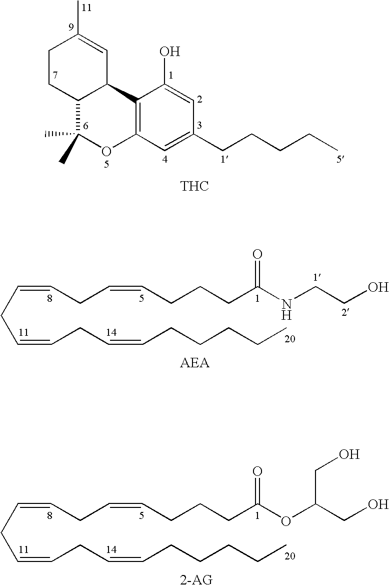

Numerous studies have demonstrated that AEA is produced as a consequence of more or less severe cell damage, sometimes caused by an excessive concentration of intracellular calcium (Hansen et al. 1998), or by macrophages and platelets during hypotensive states following hemorrhagic shock (Wagner et al. 1997) or endotoxic shock (Varga et al. 1998). Thus, endocannabinoids seem to be released to protect the body under particular conditions of physiological stress, exercising an anti-oxidising, hypotensive, immunosuppressive, anti-inflammatory and, in particular, pain-reducing action (Calignano et al. 1997).

The molecular bases of their activity have also been clarified thanks to the understanding of their metabolism, comprising the physiological paths that lead to the synthesis and inactivation of endocannabinoids. Both AEA and 2-AG are synthesised starting from membrane phospholipids and immediately released in the extracellular space to act on the same cell or on adjacent cells, but in any case in the immediate vicinity (respectively, autocrine and paracrine action); the inactivation takes place in two stages, comprising first the re-uptake of the mediator, by a transporter protein called “Anandamide membrane transporter” (AMT), and then the intracellular enzymatic hydrolysis by means of a “Fatty acid Amide Hydrolase” (FAAH).

Therefore, the four proteins of the endocannabinoid system, comprising the two receptors CB1 and CB2, FAAH and AMT, represent excellent targets for the development of new drugs to be used in different pathologies related to pain, immunosuppression, peripheral vascular problems, appetite loss or increase and motor disorders.

Therefore, the need to obtain compounds with “cannabinergic” activity, meaning by this term compounds able generically to act on the endocannabinoid system (Goutopoulos et al. 2002) is readily apparent.

However, all work completed heretofore has been aimed at the chemical modification of THC or of its analogues, or of anandamide, after its identification in animals. Whilst cannabis derivatives are generally stable compounds, anandamide is chemically unstable because, in contact with air, it is rapidly oxidised and hence degraded.

Anandamide, like 2-AG, is produced by phospholipid membrane precursors, whilst the biogenesis of THC in plants seems to originate from the union of two units, olivetol and geranyl pyrophosphate (Di Marzo et al., 2004):

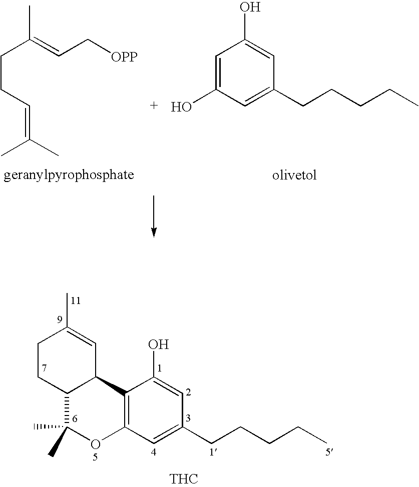

All studies conducted on THC and its congeners and on the AEA after its discovery were aimed at modifying the respective structures to obtain compounds similar to the cannabinoid receptors.

Therefore, the need is readily apparent of devising a system for synthesising compounds that are not only active on the cannabinoid receptors, but also provided with greater chemical stability.

The authors of the present invention have synthesised such compounds from a portion having aromatic characteristic, of the olivetol or modified type, whereto was linked an aliphatic chain of various lengths bearing at the end an amidic group, formed by various primary or secondary amines, or an ester residue.

### DISCLOSURE OF THE INVENTION

The present invention relates to compounds of general formula [1], chemically stable, tested in their ability to bind cannabinoid receptors and hence with potential activity in illnesses related to pain, immunosuppression, appetite loss or increase, motor disorders or peripheral vascular problems.

Therefore, an object of the present invention is a compound with the formula:

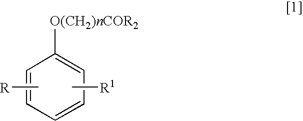

where

n varying from 1 to 15;

R represents a hydrogen atom, a halogen, a hydroxyl group or an alkyloxy group;

R₁ represents a hydrogen atom, a halogen (chlorine, bromine, iodine, fluorine), a hydroxyl group or alkyloxy group or a saturated or unsaturated alkylic chain of various lengths.

R₂ represents a primary or secondary amine (ethanolamine, propanolamine, propylamine, cyclopropylamine, cyclopropylethylamine, 2-chloroethylamine, p-hydroxyphenylamine, etc.) or an ester residue.

Preferred compounds of the invention are:

6-(3-Pentadecyl-phenoxy)-hexanoic acid (2-hydroxy-ethyl)-amide, 6-(3-Pentadecyl-phenoxy)-hexanoic acid cyclopropylamide, 6-(3-Pentadecyl-phenoxy)-hexanoic acid (4-hydroxy-phenyl)-amide, 11-(3-Pentadecyl-phenoxy)-undecanoic acid (2-hydroxy-ethyl)-amide, acid 11-(3-Pentadecyl-phenoxy)-undecanoic cyclopropylamide, 11-(3-Pentadecyl-phenoxy)-undecanoic acid (4-hydroxy-phenyl)-amide, 6-(3-hydroxy-5-pentil-phenoxy)-hexanoic acid (2-hydroxy-ethyl)-amide, 6-(3-Hydroxy-5-pentil-phenoxy)-hexanoic acid cyclopropylamide, 6-(3-hydroxy-5-pentil-phenoxy)-hexanoic acid (4-hydroxy-phenyl)amide, 11-(3-Hydroxy-5-pentil-phenoxy)-undecanoic acid (2-hydroxy-ethyl)-amide, 11-(3-hydroxy-5-pentilphenoxy) undecanoic acid cyclopropylamide, 11-(3-hydroxy-5-pentil-phenoxy)-undecanoic acid (4-hydroxy-phenyl)-amide, 16-(3-hydroxy-5-pentil-phenoxy)-hexadecanoic acid (2-hydroxy-ethyl)-amide, 16-(3-hydroxy-5-pentil-phenoxy)-hexadecanoic acid cyclopropylamide, 16-(3-hydroxy-5-pentil-phenoxy)-hexadecanoic acid (4-hydroxy-phenyl)-amide, 6-(3-hydroxy-phenoxy)-hexanoic acid (2-hydroxy-ethyl)-amide, 6-(3-hydroxy-phenoxy)-hexanoic acid cyclopropylamide, 6-(3-hydroxy-phenoxy)-hexanoic acid (4-hydroxy-phenyl)-amide, 11-(3-hydroxy-phenoxy)-undecanoic acid (2-hydroxy-ethyl)-amide, 11-(3-hydroxy-phenoxy)-undecanoic acid cyclopropylamide, 11-(3-hydroxy-phenoxy)-undecanoic acid (4-hydroxy-phenyl)-amide, 16-(3-hydroxy-phenoxy)-hexadecanoic acid (2-hydroxy-ethyl)-amide, 16-(3-hydroxy-phenoxy)-hexadecanoic acid cyclopropylamide, 16-(3-hydroxy-phenoxy)-hexadecanoic acid (4-hydroxy-phenyl)-amide, 6-(3-hydroxy-4-hexyl-phenoxy)-hexanoic acid (2-hydroxy-ethyl)-amide, 6-(3-hydroxy-4-hexyl-phenoxy)-hexanoic acid cyclopropylamide, 6-(3-hydroxy-4-hexyl-phenoxy)-hexanoic acid (4-hydroxy-phenyl)-amide, 11-(3-hydroxy-4-hexyl-phenoxy)-undecanoic acid (2-hydroxy-ethyl)-amide, 11-(3-hydroxy-4-hexyl-phenoxy)-undecanoic acid cyclopropylamide, 11-(3-hydroxy-4-hexyl-phenoxy)-undecanoic acid (4-hydroxy-phenyl)-amide, 16-(3-hydroxy-4-hexyl-phenoxy)-hexadecanoic acid (2-hydroxy-ethyl)-amide, 16-(3-hydroxy-4-hexyl-phenoxy)-hexadecanoic acid cyclopropylamide, 16-(3-hydroxy-4-hexyl-phenoxy)-hexadecanoic acid (4-hydroxy-phenyl)-amide, 6-(2-hexyl-5-hydroxy-phenoxy)-hexanoic acid (2-hydroxy-ethyl)-amide, 6-(2-hexyl-5-hydroxy-phenoxy)-hexanoic acid cyclopropylamide, 6-(2-hexyl-5-hydroxy-phenoxy)-hexanoic acid (4-hydroxy-phenyl)-amide, 11-(2-hexyl-5-hydroxy-phenoxy)-undecanoic acid (2-hydroxy-ethyl)-amide, 11-(2-hexyl-5-hydroxy-phenoxy)-undecanoic acid cyclopropylamide, 11-(2-hexyl-5-hydroxy-phenoxy)-undecanoic acid (4-hydroxy-phenyl)-amide, 16-(2-hexyl-5-hydroxy-phenoxy)-hexadecanoic acid (2-hydroxy-ethyl)-amide, 16-(2-hexyl-5-hydroxy-phenoxy)-hexadecanoic acid cyclopropylamide, 16-(2-hexyl-5-hydroxy-phenoxy)-hexadecanoic acid (4-hydroxy-phenyl)-amide.

The compounds of the invention are advantageously used for their ability to bind at least one of the two endocannabinoid receptors, and therefore as therapeutic agents, in particular for pain therapy and/or anti-inflammatory and/or anti-stress and/or anti-oxidising and/or hypotensive and/or immune suppressive therapy and/or obesity treatment and/or anti-metabolic syndrome and/or anti-spastic activity in multiple sclerosis and/or anti-cancer.

The present invention shall now be described in its non limiting examples thereof, with particular reference to the following figure:

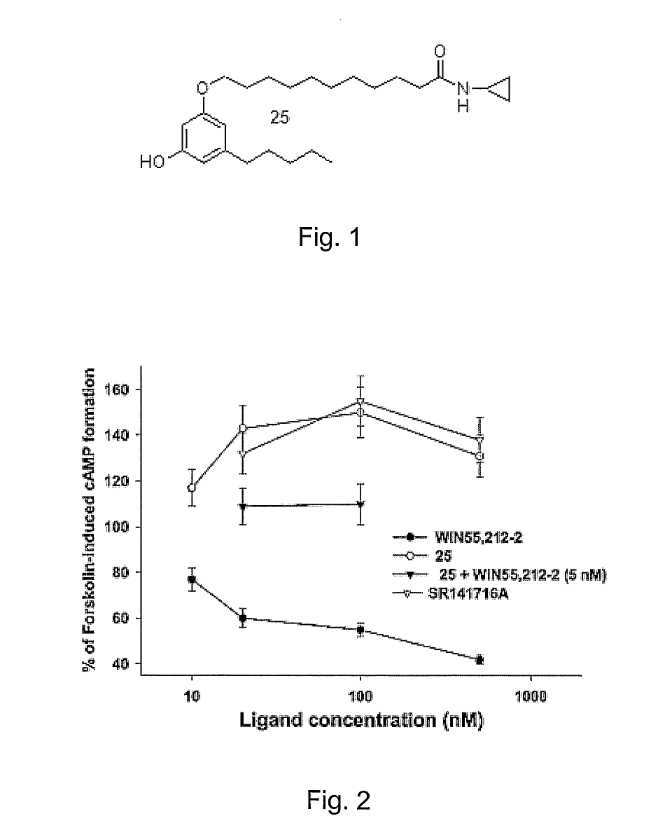

FIG. 1: Chemical structure of compound 25.

FIG. 2: Effect of compound 25 on the induced adenylate cyclase activity of forskolin. The test was conducted on mouse N18TG2 neuroblastoma cells.

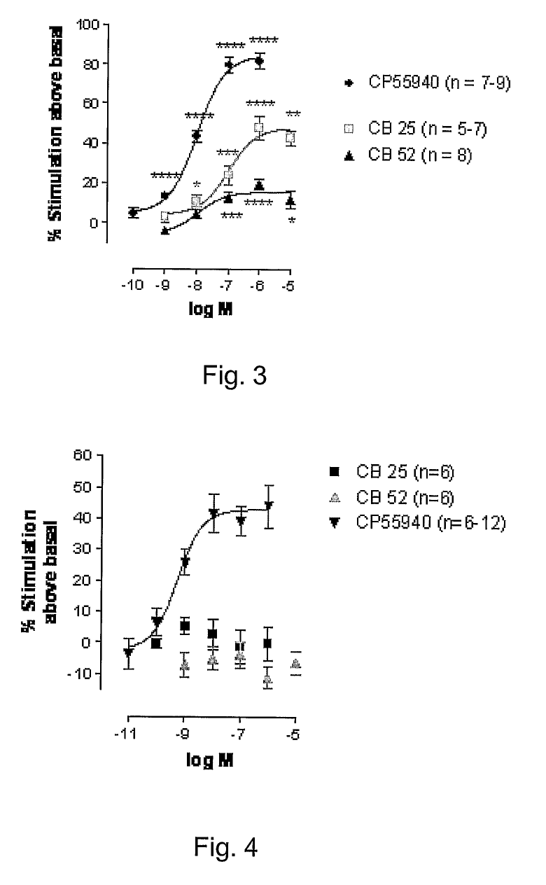

FIG. 3: Stimulation of [³⁵S]GTP-γ-S binding by compounds 25 and 52 using mouse brain membranes. The effect is compared to that of the potent CB₁ and CB₂ receptor agonist CP55940.

FIG. 4: Stimulation of [³⁵S]GTP-γ-S binding by compounds 25 and 52 using CHO cells over-expressing the human recombinant CB₂ receptor. The effect is compared to that of the potent CB₁ and CB₂ receptor agonist CP55940.

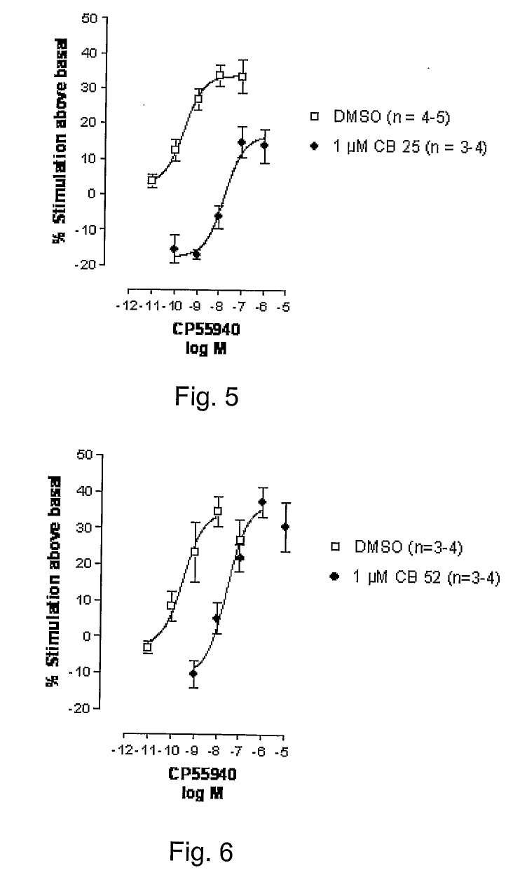

FIG. 5: Antagonism of CP55940-induced stimulation of [³⁵S]GTP-γ-S binding by compound 25 using CHO cells over-expressing the human recombinant CB₂ receptor.

FIG. 6: Antagonism of CP55940-induced stimulation of [³⁵S]GTP-γ-S binding by compound 52 using CHO cells over-expressing the human recombinant CB₂ receptor.

The compounds of the invention can be prepared by the methods described below. However, the invention is not limited to these methods, for these same compounds can also be prepared with methods known to those skilled in the art.

All compounds have as starting product a phenol, or a similar compound, which can be obtained from commercial sources or by simple organic chemistry reactions, which is made to react with a bromoester:

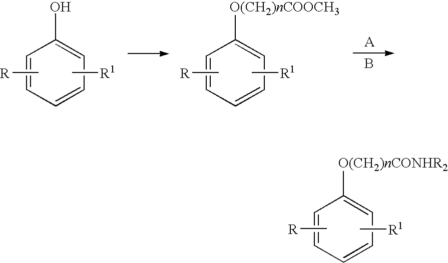

The esters thus obtained are transformed into the final amides by two general methods.

Method A. the methyl ester is placed to react, under agitation and in inert atmosphere, with a moderate excess of amine, used as solvent, and the reaction mixture maintained at 120-130° C. for 5 hours. The reaction mixture was then poured in water, extracted with chloroform or ethyl acetate and the collected organic extracts were washed with saturated solution of ammonium chloride, dried, filtered and evaporated. The reaction raw material thus obtained was purified by chromatography on silicon gel.

Method B. the methyl ester is dissolved in methanol and, under agitation, a slight excess of aqueous solution of sodium hydroxide is added; the solution is maintained in reflux for about 3 hours. The solution, cooled in ice bath, is acidified and then extracted with suitable organic solvent. The reunited organic phases, dried, filtered and evaporated, provide a reaction blank that is purified by chromatography on silica gel, with chloroform/methanol mixture of suitable concentration.

The acid thus obtained (1 eq.) is dissolved in anhydrous chloromethane or acetonitrile, according to solubility, with the amine (1.5 eq.) and to the solution, under agitation and in inert atmosphere, are added in order, at the temperature of 0° C., hydroxybenzotriazole (1.2 eq., in successive portion) and, drop by drop, a solution of CMC (1.5 eq.) in anhydrous dichloromethane. The mixture is left all night at ambient temperature and then washed with hydrochloric acid 1N, then with an aqueous solution of sodium bicarbonate at 5% and dried on anhydrous sodium sulphate. The organic phase, filtered and evaporated, provides the reaction blanks that are purified by chromatography on silicon gel with appropriate eluent mixtures.

At ambient temperature, the compounds are in the solid state and stable for a long time, unlike the natural mediator anandamide.

### EXAMPLES

6-(3-Pentadecyl-phenoxy)-hexanoic acid (2-hydroxy-ethyl)-amide (15). White solid (CHCl₃/MeOH=47/3) (80% yield): m.p. 62.7° C. (M). ¹H NMR (CDCl₃) δ (ppm): 7.14-7.10 (m, 1H), 6.75-6.66 (m, 3H), 5.95 (s br, 1H), 3.93 (t, 2H, J=6.3 Hz), 3.70 (t, 2H, J=4.9 Hz), 3.44-3.36 (m, 2H), 2.65 (s br, 1H), 2.54 (t, 2H, J=7.7 Hz), 2.22 (t, 2H, J=7.3 Hz), 1.81-1.46 (mm, 8H), 1.44-1.24 (mm, 24H) 0.86 (t, 3H, J=6.2 Hz). Anal. (C₂₉H₅₁NO₃) C, H, N.

6-(3-Pentadecyl-phenoxy)-hexanoic acid cyclopropylamide (16). White solid (CHCl₃, recrystallized from acetone/ethyl ether) (50% yield): m.p. 67.8° C. (M). H NMR (CDCl₃) δ (ppm): 6.65 (t, 1H, J=7.7 Hz), 6.50 (s br, 1H), 6.26-6.17 (m, 3H), 3.46 (t, 2H, J=6.3 Hz), 2.28-2.18 (m, 1H), 2.08 (t, 2H, J=7.5 Hz), 1.69 (t, 2H, J=7.3 Hz), 1.36-1.03 (mm, 8H), 1.0-0.79 (mm, 24H), 0.40 (t, 3H, J=6.3 Hz), 0.24-0.15 (m, 2H), 0.11-0.02 (m, 2H). MS m/z: 458 [M+1]⁺ (100). Anal. (C₃₀H₅₁NO₂) C, H, N.

6-(3-Pentadecyl-phenoxy)-hexanoic acid (4-hydroxy-phenyl)-amide (17). White solid (CHCl₃/MeOH=47/3) (70% yield): m.p. 103.2° C. (M). H NMR (CDCl₃) δ (ppm): 7.31 (half of AB_{q}, 2H, J=8.7 Hz), 7.20-7.12 (m, 2H), 7.00 (s br, 1H), 6.79-6.68 (m, 4H), 3.95 (t, 2H, J=7.1 Hz), 3.92 (s br, 1H), 2.55 (t, 2H, J=7.6 Hz), 2.36 (t, 2H, J=7.0 Hz), 1.80-1.69 (m, 4H), 1.63-1.53 (m, 4H), 1.25-1.18 (mm, 24H), 0.87 (t, 3H, J=6.6 Hz). MS m/z: 510 [M+1]⁺, 1019 [2M+1]⁺ (100). Anal. (C₃₃H₅₁NO₃) C, H, N.

11-(3-Pentadecyl-phenoxy)-undecanoic acid (2-hydroxy-ethyl)-amide (18). White solid (CHCl₃/MeOH=47/3) (75% yield): m.p. 83-85° C. (K). H NMR (CDCl₃) δ (ppm): 7.18-7.11 (m, 1H), 6.74-6.67 (m, 3H), 5.85 (s br, 1H), 3.92 (t, 2H, J=6.4 Hz), 3.71 (t, 2H, J=4.7 Hz), 3.44-3.36 (m, 2H), 2.60 (s br, 1H), 2.55 (t, 2H, J=7.5 Hz), 2.18 (t, 2H, J=7.5 Hz), 1.78-1.51 (mm, 6H), 1.28-1.24 (mm, 36H), 0.86 (t, 3H, J=6.3 Hz). Anal. (C₃₄H₆₁NO₃) C, H, N.

11-(3-Pentadecyl-phenoxy)-undecanoic acid cyclopropylamide (19). White needles (CHCl₃, recrystallized from acetone) (91% yield): m.p. 82.0° C. (K). H NMR (CDCl₃) δ (ppm): 7.15 (t, 1H, J=8.1 Hz), 6.75-6.67 (m, 3H), 5.46 (s br, 1H), 3.92 (t, 2H, J=6.5 Hz), 2.71-2.64 (m, 1H), 2.55 (t, 2H, J=7.6 Hz), 2.09 (t, 2H, J=7.5 Hz), 1.78-1.41 (mm, 10H), 1.27-1.24 (mm, 32H), 0.86 (t, 3H, J=6.2 Hz), 0.80-0.70 (m, 2H), 0.55-0.46 (m, 2H). MS m/z: 1077 [2M+Na]⁺ (100). Anal. (C₃₅H₆₁NO₂) C, H, N.

11-(3-Pentadecyl-phenoxy)-undecanoic acid (4-hydroxy-phenyl)-amide (20). White solid (CHCl3/MeOH=47/3) (86% yield): m.p. 107.0° C. (M). H NMR (CDCl₃) δ (ppm): 7.33 (half of AB_{q}, 2H, J=7.5 Hz), 7.21-7.12 (m, 2H), 7.01 (s br, 1H), 6.97-6.54 (m, 4H), 3.93 (t, 2H, J=6.5 Hz), 2.56 (t, 2H, J=7.7 Hz), 2.35 (t, 2H, J=7.4 Hz), 1.79-1.60 (m, 8H), 1.30-1.22 (mm, 34H), 0.87 (t, 3H, J=6.6 Hz). MS: 579 [M]⁺, 1159 [2M+1]⁺ (100). Anal. (C₃₈H₆₁NO₃) C, H, N.

6-(3-Hydroxy-5-pentyl-phenoxy)-hexanoic acid (2-hydroxy-ethyl)-amide (21). Pink oil (ethyl acetate/MeOH=46/4) (65% yield). H NMR (Acetone-d₆) δ (ppm): 8.43 (s, 1H), 7.33 (s br, 1H), 6.25-6.21 (m, 3H), 4.18 (s br, 1H), 3.86 (t, 2H, J=6.3 Hz), 3.60-3.57 (m, 2H), 3.37-3.28 (m, 2H), 2.44 (t, 2H, J=7.6 Hz), 2.22 (t, 2H, J=7.3 Hz), 1.70-1.29 (mm, 12H), 0.85 (t, 3H, J=6.5 Hz). Anal. (C₁₉H₃₁NO₄) C, H, N.

6-(3-Hydroxy-5-pentyl-phenoxy)-hexanoic acid cyclopropylamide (22); Pale yellow oil (CHCl₃/MeOH=48/2) (60%). H NMR (CDCl₃) δ (ppm): 7.04 (s br, 1H), 6.29-6.24 (m, 3H), 5.87 (s br, 1H), 3.86 (t, 2H, J=6.2 Hz), 2.74-2.65 (m, 1H), 2.46 (t, 2H, J=7.6 Hz), 2.14 (t, 2H, J=7.3 Hz), 1.75-1.24 (mm, 12H), 0.86 (t, 3H, J=6.5 Hz), 0.78-0.69 (m, 2H), 0.56-0.47 (m, 2H). Anal. (C₂₀H₃₁NO₃) C, H, N.

6-(3-Hydroxy-5-pentyl-phenoxy)-hexanoic acid (4-hydroxy-phenyl)amide (23). White solid (CHCl₃/MeOH=45/5) (75% yield): m.p. 69.2° C. (M). H NMR (CDCl₃) δ (ppm): 7.33 (half of AB_{q}, 2H, J=8.7 Hz), 7.02 (s br, 1H), 6.80 (half of AB_{q}, 2H, J=8.7 Hz), 6.29-6.21 (m, 3H), 4.95 (s br, 1H), 3.92 (t, 2H, J=6.2 Hz), 2.49 (t, 2H, J=7.6 Hz), 2.36 (t, 2H, J=7.2 Hz), 1.83-1.76 (m, 4H), 1.61-1.53 (m, 4H), 1.33-1.25 (mm, 4H), 0.88 (t, 3H, J=6.5 Hz). MS m/z: 386 [M+1]⁺ (100), 771 [2M+1]⁺, 793 [2M+Na]⁺. Anal. (C₂₃H₃₁NO₄) C,H,N.

11-(3-Hydroxy-5-pentyl-phenoxy)-undecanoic acid (2-hydroxy-ethyl)-amide (24). Pasty pink solid (ethyl acetate/MeOH=44/6) (70% yield): m.p. 55-56° C. (K). H NMR (DMSO) δ (ppm): 9.34 (s, 1H), 7.68 (s br, 1H), 6.13-6.09 (m, 3H), 4.57 (t, 1H, J=5.3 Hz), 3.82 (t, 2H, J=6.3 Hz), 3.39-3.30 (m, 2H), 3.11-3.03 (m, 2H), 2.39 (t, 2H, J=7.5 Hz), 2.02 (t, 2H, J=7.2 Hz), 1.63-1.45 (mm, 6H), 1.41-1.23 (mm, 16H), 0.83 (t, 3H, J=6.4 Hz). Anal. (C₂₄H₄₁NO₄) C, H, N.

11-(3-Hydroxy-5-pentyl-phenoxy)-undecanoic acid cyclopropylamide (25). White solid (CHCl₃/MeOH=47/3) (61% yield): m.p. 70.7° C. (M). H NMR (CDCl₃) δ (ppm): 6.98 (s br, 1H), 6.28-6.26 (m, 3H), 5.79 (s br, 1H), 3.88 (t, 2H, J=6.3 Hz), 2.72-2.67 (m, 1H), 2.47 (t, 2H, J=7.6 Hz), 2.12 (t, 2H, J=7.5 Hz), 1.75-1.49 (mm, 8H), 1.28-1.26 (mm, 14H), 0.87 (t, 3H, J=6.4 Hz), 0.79-0.70 (m, 2H), 0.55-0.43 (m, 2H). Anal. (C₂₅H₄₁NO₃) C, H, N.

11-(3-Hydroxy-5-pentyl-phenoxy)-undecanoic acid (4-hydroxy-phenyl)-amide (26). Pasty white solid (CHCl₃/MeOH=47/3) (65% yield). H NMR (CDCl₃) δ (ppm): 7.28 (half of AB_{q}, 2H, J=9.0 Hz), 7.15 (s br, 1H), 6.74 (half of AB_{q}, 2H, J=8.8 Hz), 6.29-6.25 (m, 3H), 3.90 (t, 2H, J=6.4 Hz), 2.50 (t, 2H, J=7.6 Hz), 2.32 (t, 2H, J=7.4 Hz), 1.73-1.53 (m, 8H), 1.33-1.29 (m, 14H), 0.87 (t, 3H, J=6.6 Hz). MS m/z: 456 [M+1]⁺ (100), 911 [2M+1]⁺, 933 [2M+Na]⁺. Anal. (C₂₈H₄₁NO₄) C, H, N.

16-(3-Hydroxy-5-pentyl-phenoxy)-hexadecanoic acid (2-hydroxy-ethyl)-amide (27). White solid (ethyl acetate/MeOH=50/5; recrystallized from ethyl acetate) (57% yield): m.p. 84-85° C. (K). H NMR (DMSO) δ (ppm): 9.17 (s, 1H), 7.72 (s br, 1H), 6.17-6.13 (m, 3H), 4.61 (t, 1H, J=5.1 Hz), 3.86 (t, 2H, J=6.1 Hz), 3.40-3.33 (m, 2H), 3.15-3.09 (m, 2H), 2.42 (t, 2H, J=7.3 Hz), 2.06 (t, 2H, J=7.2 Hz), 1.66-1.52 (mm, 8H), 1.48-1.25 (mm, 24H), 0.87 (t, 3H, J=6.3 Hz). Anal. (C₂₉H₅₁NO₄) C, H, N.

16-(3-Hydroxy-5-pentyl-phenoxy)-hexadecanoic acid cyclopropylamide (28). White solid (CHCl₃/MeOH=48/2) (88.5% yield): m.p. 91.0° C. (M). H NMR (CDCl₃) δ (ppm): 6.29-6.24 (m, 3H), 5.52 (s br, 1H), 3.89 (t, 2H, J=6.4 Hz), 2.72-2.67 (m, 1H), 2.48 (t, 2H, J=7.6 Hz), 2.11 (t, 2H, J=7.5 Hz), 1.76-1.66 (mm, 8H), 1.57-1.24 (mm, 24H), 0.87 (t, 3H, J=6.5 Hz), 0.80-0.71 (m, 2H), 0.55-0.42 (m, 2H). Anal. (C₃₀H₅₁NO₃) C, H, N.

16-(3-Hydroxy-5-pentyl-phenoxy)-hexadecanoic acid (4-hydroxy-phenyl)-amide (29). White solid (CHCl₃/MeOH=46/4) (83.0% yield): m.p. 89.5° C. (M). H NMR (DMSO) δ (ppm): 9.50 (s, 1H), 9.30 (s, 1H), 9.12 (s br, 1H), 7.29 (half of AB_{q}, 2H, J=8.4 Hz), 6.63 (half of AB_{q}, 2H, J=8.6 Hz), 6.12-6.07 (m, 3H), 3.80 (t, 2H, J=6.3 Hz), 2.37 (t, 2H, J=7.4 Hz), 2.14 (t, 2H, J=7.6 Hz), 1.70-1.15 (mm, 32H), 0.81 (t, 3H, J=6.4 Hz). Anal. (C₃₃H₅₁NO₄) C, H, N.

6-(3-Hydroxy-phenoxy)-hexanoic acid (2-hydroxy-ethyl)-amide (30). Pale pink oil (ethyl acetate/MeOH=50/2) (50.0% yield). H NMR (CDCl₃) δ (ppm): 8.70 (s br, 1H), 7.41 (s br, 1H), 7.02 (t, 1H, J=8.3 Hz), 6.97-6.33 (m, 3H), 4.31 (s br, 1H), 3.87 (t, 2H, J=6.3 Hz), 3.62-3.57 (m, 2H), 3.35-3.27 (m, 2H), 2.23 (t, 2H, J=7.2 Hz), 1.74-1.58 (m, 4H), 1.50-1.43 (m, 2H). MS m/z: 268 [M+1]⁺ (100), 290 [M+Na]⁺. Anal. (C₁₄H₂₁NO₄) C, H, N.

6-(3-Hydroxy-phenoxy)-hexanoic acid cyclopropylamide (31). White needles (CHCl₃/MeOH=47/3) (86% yield): 102.5° C. (M). H NMR (CDCl₃) δ (ppm): 7.12-7.04 (m, 1H), 6.43-6.40 (m, 3H), 6.11 (s, 1H), 5.63 (s br, 1H), 3.90 (t, 2H, J=6.1 Hz), 2.74-2.67 (m, 1H), 2.14 (t, 2H, J=7.3 Hz), 1.82-1.62 (mm, 4H), 1.55-1.37 (m, 2H), 0.80-0.70 (m, 2H), 0.50-0.47 (m, 2H). MS: 264 [M+1]⁺, 527 [2M+1]⁺, 549 [2M+Na]⁺ (100). Anal. (C₁₅H₂₁NO₃) C, H, N.

6-(3-Hydroxy-phenoxy)-hexanoic acid (4-hydroxy-phenyl)-amide (32). Pale pink solid (CHCl₃/MeOH=45/5 ; recrystallized from ethyl acetate) (88.0% yield): 123.1° C. (M). H NMR (DMSO) δ (ppm): 9.53 (s, 1H), 9.26 (s, 1H), 9.05 (s, 1H), 7.31 (half of AB_{q}, 2H, J=8.8 Hz), 6.95 (t, 1H, J=8.1 Hz), 6.64 (half of AB_{q}, 2H, J=8.7 Hz), 6.31-6.27 (m, 3H), 3.85 (t, 2H, J=6.3 Hz), 2.23 (t, 2H, J=7.2 Hz), 1.74-1.53 (m, 4H), 1.46-1.36 (m, 2H). Anal. (C₁₈H₂₁NO₄) C, H, N.

11-(3-Hydroxy-phenoxy)-undecanoic acid (2-hydroxy-ethyl)-amide (33). Cream solid (Ethyl acetate/MeOH=50/4) (34% yield): m.p. 72.1° C. (M). H-NMR (CDCl₃) δ (ppm): 7.10 (t, 1H, J=8.0 Hz), 6.44-6.38 (m, 3H), 6.15 (s br, 1H), 5.96 (s br, 1H), 3.92 (t, 2H, J=6.2 Hz), 3.74-3.70 (m, 2H), 3.46-3.38 (m, 2H), 2.58 (s br, 1H), 2.19 (t, 2H, J=7.5 Hz), 2.02-1.56 (mm, 6H), 1.43-1.24 (mm, 10H). MS m/z: 338 [M+1]⁺, 360 [M+Na]⁺, 697 [2M+Na]⁺(100). Anal. (C₁₉H₃₁NO₄) C, H, N.

11-(3-Hydroxy-phenoxy)-undecanoic acid cyclopropilamide (34). White solid (Ethyl acetate) (90% yield): m.p. 92.8° C. (M). H NMR (CDCl₃) δ (ppm): 7.08 (t, 1H, J=8.2 Hz), 6.50 (s br, 1H), 6.47-6.39 (m, 3H), 5.60 (s br, 1H), 3.91 (t, 2H, J=6.3 Hz), 2.76-2.66 (m, 1H), 2.11 (t, 2H, J=7.2 Hz), 1.79-1.60 (mm, 4H), 1.46-1.27 (mm, 12H), 0.80-0.70 (m, 2H), 0.48-0.42 (m, 2H). MS m/z: 334 [M+1]⁺, 667 [2M+1]⁺(100), 689 [2M+Na]⁺. Anal. (C₂₀H₃₁NO₃) C, H, N.

11-(3-Hydroxy-phenoxy)-undecanoic acid (4-hydroxy-phenyl)-amide (35). White solid (CHCl₃/MeOH=45/5) (79% yield): m.p. 121.2° C. (M). H-NMR (DMSQ)δ (ppm): 9.50 (s, 1H), 9.26 (s, 1H), 9.05 (s, 1H), 7.30 (half of AB_{q}, 2H, J=8.7 Hz), 6.98 (t, 1H, J=8.0 Hz), 6.63 (half of AB_{q}, 2H, J=8.6 Hz), 6.31-6.26 (m, 3H), 3.83 (t, 2H, J=6.4 Hz), 2.19 (t, 2H, J=7.3 Hz), 1.62-1.56 (m, 4H), 1.53-1.24 (mm, 12H). Anal. (C₂₃H₃₁NO₄) C, H, N.

16-(3-Hydroxy-phenoxy)-hexadecanoic acid (2-hydroxy-ethyl)-amide (36). White solid (Ethyl acetate/MeOH=50/1) (35% yield): m.p. 94.5° C. (M). H NMR (DMSO) δ (ppm): 9.51 (s, 1H), 7.91 (s br, 1H), 7.44 (t, 1H, J=8.2 Hz), 6.81-6.75 (m, 3H), 4.80 (t, 1H, J=5.5 Hz), 4.33 (t, 2H, J=6.4 Hz), 3.94-3.85 (m, 2H), 3.66-3.57 (m, 2H), 2.54 (t, 2H, J=7.0 Hz), 2.18-2.10 (m, 4H), 2.00-1.70 (mm, 22H). Anal. (C₂₄H₄₁NO₄) C, H, N.

16-(3-Hydroxy-phenoxy)-hexadecanoic acid cyclopropylamide (37). White bright solid (Ethyl acetate/MeOH=50/4; recrystallized Ethyl acetate) (78% yield): m.p. 105.6° C. (M). H NMR (DMSO) δ (ppm): 9.25 (s, 1H), 7.73 (s br, 1H), 6.98 (t, 1H, J=7.9 Hz), 6.31-6.26 (m, 3H), 3.83 (t, 2H, J=6.4 Hz), 2.58-2.51 (m, 1H), 1.94 (t, 2H, J=7.3 Hz), 1.64-1.59 (m, 2H), 1.41-1.20 (mm, 24H), 0.58-0.49 (m, 2H), 0.40-0.26 (m, 2H). Anal. (C₂₅H₄₁NO₃) C, H, N.

16-(3-Hydroxy-phenoxy)-hexadecanoic acid (4-hydroxy-phenyl)-amide (38). White solid (CHCl₃/MeOH=50/5) (71.5% yield): 123.6° C. (M). H NMR (DMSO) δ (ppm): 9.57 (s, 1H), 9.32 (s,1H), 9.05 (s, 1H), 7.36 (half of AB_{q}, 2H, J=8.6 Hz), 7.04 (t, 1H, J=7.9 Hz), 6.69 (half of AB_{q}, 2H, J=8.6 Hz), 6.37-6.32 (m, 3H), 3.89 (t, 2H, J=6.3 Hz), 2.24 (t, 2H, J=7.3 Hz), 1.69-1.57 (m, 4H), 1.50-1.27 (mm, 22H). Anal. (C₂₈H₄₁NO₄) C, H, N.

6-(3-Hydroxy-4-hexyl-phenoxy)-hexanoic acid (2-hydroxy-ethyl)-amide (39). Pale yellow solid (CHCl₃/MeOH=46/4) (55% yield): m.p. 71.2° C. (M). H-NMR (Acetone-d₆) δ (ppm): 8.42 (s, 1H), 7.42 (s br, 1H), 6.93 (d, 1H, J=8.0 Hz), 6.45 (d, 1H, J=2.0 Hz), 6.32 (dd, 1H, J=2.0 Hz, J=8.0 Hz), 4.30 (t, 1H, J=5.2 Hz), 3.87 (t, 2H, J=6.3 Hz), 3.67-3.59 (m, 2H), 3.40-3.31 (m, 2H), 2.54 (t, 2H, J=7.5 Hz), 2.27 (t, 2H, J=7.3 Hz), 1.76-1.65 (m, 4H), 1.61-1.42 (mm, 4H), 1.32-1.30 (m, 6H), 0.88 (t, 3H, J=6.1 Hz). MS m/z: 352 [M+1]⁺, 374 [M+Na]⁺ (100), 725 [2M+Na]⁺. Anal. (C₂₀H₃₃NO₄) C, H, N.

6-(3-Hydroxy-4-hexyl-phenoxy)-hexanoic acid cyclopropylamide (40). White bright solid (Ethyl acetate; recrystallized from n.hexane) (70% yield): m.p. 134.2° C. (M). H-NMR 300 MHz (DMSO) δ (ppm): 9.11 (s, 1H), 7.80 (d, 1H, J=3.6 Hz)), 6.85 (d, 1H, J=8.4 Hz), 6.31 (d, 1H, J=2.4 Hz), 6.24 (dd, 1H, J=2.4 Hz, J=8.4 Hz), 3.80 (t, 2H, J=6.6 Hz), 2.61-2.53 (m, 1H), 2.42 (t, 2H, J=7.3 Hz), 2.00 (t, 2H, J=7.1 Hz), 1.68-1.60 (m, 2H), 1.55-1.41 (m, 4H), 1.37-1.23 (mm, 8H), 0.83 (t, 3H, J=6.6 Hz), 0.60-0.53 (m, 2H), 0.36-0.31 (m, 2H). MS m/z: 348 [M+1]⁺ (100), 695 [2M+1]⁺. Anal. (C₂₁H₃₃NO₃) C, H, N.

6-(3-Hydroxy-4-hexyl-phenoxy)-hexanoic acid (4-hydroxy-phenyl)-amide (41). White solid (CHCl₃/MeOH=47/3) (79.5% yield): m.p. 128.4° C. H-NMR (Acetone-d₆) δ (ppm): 8.82 (s, 1H), 8.00 (s br, 1H), 7.43 (half of AB_{q}, 2H, J=8.6 Hz), 6.92 (half of AB_{q}, 2H, J=8.6 Hz), 6.73 (d, 1H, J=8.8 Hz), 6.40 (d, 1H, J=2.7 Hz), 6.31 (dd, 1H, J=2.5 Hz, J=8.7 Hz), 3.87 (t, 2H, J=6.4 Hz), 2.51 (t, 2H, J=7.4 Hz), 2.32 (t, 2H, J=7.2 Hz), 1.77-1.64 (m, 6H), 1.55-1.44 (m, 4H), 1.40-1.27 (m, 4H), 0.85 (t, 3H, J=6.6 Hz). Anal. (C₂₄H₃₃NO₄) C, H, N.

11-(3-Hydroxy-4-hexyl-phenoxy)-undecanoic acid (2-hydroxy-ethyl)-amide (42). White solid (Ethyl acetate/MeOH=50/4) (43% yield): m.p. 85.3° C. (M). H NMR (CDCl₃) δ (ppm): 6.96 (d, 1H, J=9.0 Hz), 6.40-6.35 (m, 2H), 5.96 (s br, 1H), 3.89 (t, 2H, J=6.2 Hz), 3.73 (t, 2H, J=5.0 Hz), 3.46-3.39 (m, 2H), 2.51 (t, 2H, J=7.6 Hz), 2.21 (t, 2H, J=7.5 Hz), 1.76-1.52 (mm, 8H), 1.45-1.22 (mm, 16H), 0.87 (t, 3H, J=6.4 Hz). MS m/z: 422 [M+1]⁺, 444 [M+Na]⁺ (100). Anal. (C₂₅H₄₃NO₄) C, H, N.

11-(3-Hydroxy-4-hexyl-phenoxy)-undecanoic acid cyclopropylamide (43). White bright solid (Ethyl acetate; recrystallized from ethyl acetate/n.hexane) (81% yield): 112.3° C. (M). H-NMR 300 MHz (DMSO) δ (ppm): 9.10 (s, 1H), 7.77 (s br, 1H), 6.85 (d, 1H, J=8.4 Hz), 6.30 (d, 1H, J=2.4 Hz), 6.24 (dd, 1H, J=2.4 Hz, J=8.0 Hz), 3.81 (t, 2H, J=6.4 Hz), 2.59-2.53 (m, 1H), 2.39 (t, 2H, J=7.4 Hz), 1.96 (t, 2H, J=7.4 Hz), 1.65-1.58 (m, 2H), 1.46-1.38 (m, 4H), 1.34-1.23 (mm, 18H), 0.83 (t, 3H, J=6.4 Hz), 0.57-0.53 (m, 2H), 0.34-0.31 (m, 2H). MS m/z: 418 [M+1]⁺, 440 [M+Na]⁺ (100). Anal. (C₂₆H₄₃NO₃) C, H, N.

11-(3-Hydroxy-4-hexyl-phenoxy)-undecanoic acid (4-hydroxy-phenyl)-amide (44). White solid (CHCl₃/MeOH=48/2) (88.0% yield): m.p. 121.2° C. (M). H-NMR (DMSO) δ (ppm): 9.48 (s, 1H), 9.04 (s, 1H), 9.02 (s, 1H), 7.30 (half of AB_{q}, 2H, J=8.8 Hz), 6.84 (d, 1H, J=8.1 Hz), 6.63 (half of AB_{q}, 2H, J=8.8 Hz), 6.29 (d, 1H, J=2.1 Hz), 6.23 (dd, 1H, J=2.3 Hz, J=8.2 Hz), 3.80 (t, 2H, J=6.4 Hz), 2.38 (t, 2H, J=7.4 Hz), 2.20 (t, 2H, J=7.4 Hz), 1.65-1.40 (mm, 10H), 1.37-1.22 (mm, 14H), 0.82 (t, 3H, CH₃, J=6.4 Hz). Anal. (C₂₉H₄₃NO₄) C, H, N.

16-(3-Hydroxy-4-hexyl-phenoxy)-hexadecanoic acid (2-hydroxy-ethyl)-amide (45). White solid (Ethyl acetate/MeOH=50/1) (50% yield): m.p. 96.4° C. H-NMR (DMSO) δ (ppm): 9.14 (s, 1H), 7.73 (s br, 1H), 6.88 (d, 1H, J=8.3 Hz), 6.34 (d, 1H, J=1.7 Hz), 6.27 (dd, 1H, J=8.3 Hz, J=1.7 Hz), 4.63 (t, 1H, J=5.2 Hz), 3.84 (t, 2H, J=6.2 Hz), 3.57-3.40 (m, 2H), 3.15-3.06 (m, 2H), 2.43 (t, 2H, J=7.2 Hz), 2.06 (t, 2H, J=7.3 Hz), 1.70-1.48 (mm, 8H), 1.44-1.25 (mm, 26H), 0.87 (t, 3H, J=5.8 Hz). Anal. (C₃₀H₅₃NO₄) C, H, N.

16-(3-Hydroxy-4-hexyl-phenoxy)-hexadecanoic acid cyclopropylamide (46). White bright solid (Ethyl acetate/MeOH=50/2; recrystallized from ethyl acetate) (94.5% yield): m.p. 114.3° C. (M). H-NMR (DMSO) δ (ppm): 9.15 (s, 1H), 7.80 (s br, 1H), 6.82 (d, 1H, J =8.1 Hz), 6.34 (d, 1H, J=2.6 Hz), 6.20 (dd, 1H, J=2.4 Hz, J=8.3 Hz), 3.79 (t, 2H, J=6.3 Hz), 2.55-2.48 (m, 1H), 2.37 (t, 2H, J=7.4 Hz), 1.94 (t, 2H, J=7.3 Hz), 1.62-1.55 (m, 4H), 1.41-1.20 (mm, 30H), 0.82 (t, 3H, J=6.0 Hz), 0.58-0.49 (m, 2H), 0.34-0.27 (m, 2H). Anal. (C₃₁H₅₃NO₃) C, H, N.

16-(3-Hydroxy-4-hexyl-phenoxy)-hexadecanoic acid (4-hydroxy-phenyl)-amide (47). White solid (CHCl₃/MeOH=45/5) (81% yield): m.p. 133.2° C. (M). H-NMR (DMSO) δ (ppm): 9.50 (s, 1H), 9.06 (s, 1H), 9.04 (s, 1H), 7.30 (half of AB_{q}, 2H, J=8.5 Hz), 6.83 (d, 1H, J=8.0 Hz), 6.62 (half of AB_{q}, 2H, J=8.5 Hz), 6.28 (s, 1H), 6.22 (d, 1H, J=8.1 Hz), 3.80 (t, 2H, J=6.2 Hz), 2.38 (t, 2H, J=7.3 Hz), 2.18 (t, 2H, J=7.1 Hz), 1.61-1.21 (mm, 34H), 0.81 (t, 3H, J=6.4 Hz). Anal. (C₃₄H₅₃NO₄) C, H, N.

6-(2-Hexyl-5-hydroxy-phenoxy)-hexanoic acid (2-hydroxy-ethyl)-amide (48). Pale yellow thick oil (CHCl₃/MeOH=46/4) (50% yield). H-NMR (Acetone-d₆) δ (ppm): 8.23 (s, 1H), 7.27 (s br, 1H), 6.88 (d, 1H, J=8.0 Hz), 6.42 (d, 1H, J=2.0 Hz), 6.35-6.31 (m, 1H), 4.08 (s br, 1H), 3.91 (t, 2H, J=6.3 Hz), 3.61-3.58 (m, 2H), 3.36-3.28 (m, 2H), 2.50 (t, 2H, J=7.4 Hz), 2.25 (t, 2H, J=7.2 Hz), 1.85-1.67 (mm, 4H), 1.63-1.52 (m, 4H), 1.49-1.31 (mm, 6H), 0.88 (t, 3H, J=6.4 Hz). MS m/z: 353 [M+D]⁺, 374 [M+Na]⁺, 725 [2M+Na]⁺ (100). Anal. (C₂₀H₃₃NO₄) C, H, N.

6-(2-Hexyl-5-hydroxy-phenoxy)-hexanoic acid cyclopropylamide (49). Pale yellow thick oil (Ethyl acetate) (80% yield). H-NMR 300 MHz (DMSO) δ (ppm): 9.06 (s, 1H), 7.80 (d, 1H, J=3.9 Hz)), 6.81 (d, 1H, J=8.1 Hz), 6.30 (d, 1H, J=2.3 Hz), 6.21 (dd, 1H, J=2.2 Hz, J=8.1 Hz), 3.82 (t, 2H, J=6.2 Hz), 2.61-2.52 (m, 1H), 2.38 (t, 2H, J=7.1 Hz), 2.00 (t, 2H, J=7.1 Hz), 1.71-1.62 (m, 2H), 1.56-1.47 (m, 2H), 1.42-1.34 (m, 4H), 1.27-1.23 (mm, 6H), 0.82 (t, 3H, J=6.6 Hz), 0.58-0.52 (m, 2H), 0.35-0.30 (m, 2H). MS m/z: 348 [M+1]⁺ (100), 695 [2M+1]⁺. Anal. (C₂₁H₃₃NO₃) C, H, N.

6-(2-Hexyl-5-hydroxy-phenoxy)-hexanoic acid (4-hydroxy-phenyl)-amide (50). White solid (CHCl₃/MeOH=47/3) (63% yield): m.p. 106.0° C. (M). H-NMR (Acetone-d₆) δ (ppm): 9.52 (s, 1H), 9.04 (s, 1H), 9.02 (s, 1H), 7.30 (half of AB_{q}, 2H, J=8.8 Hz), 6.80 (d, 1H, J=8.1 Hz), 6.62 (half of AB_{q}, 2H, J=8.7 Hz), 6.28 (s, 1H), 6.20 (d, 1H, J=8.0 Hz), 3.83 (t, 2H, J=6.0 Hz), 2.36 (t, 2H, J=7.3 Hz), 2.23 (t, 2H, J=7.1 Hz), 1.70-1.57 (mm, 10H), 1.53-1.20 (m, 4H), 0.81 (t, 3H, J=6.7 Hz). Anal. (C₂₄H₃₃NO₄) C, H, N.

11-(2-Hexyl-5-hydroxy-phenoxy)-undecanoic acid (2-hydroxy-ethyl)-amide (51). Pale yellow solid (Ethyl acetate/MeOH=50/4) (25% yield): m.p. 75.4° C. (M). H NMR (CDCl₃) δ (ppm): 6.90 (d, 1H, J=7.9 Hz), 6.38-6.36 (d, 1H, J=2.0 Hz), 6.31 (dd, 1H, J=7.9 Hz, J=2.0 Hz), 6.08 (s br, 1H), 3.87 (t, 2H, J=6.3 Hz), 3.70 (t, 2H, J=4.9 Hz), 3.43-3.36 (m, 2H), 2.48 (t, 2H, J=7.5 Hz), 2.17 (t, 2H, J=7.6 Hz), 1.81-1.68 (m, 2H), 1.63-1.43 (mm, 4H), 1.40-1.27 (mm, 18H), 0.86 (t, 3H, J=6.3 Hz). MS m/z: 422 [M+1]⁺ (100), 444 [M+Na]⁺, 843 [2M+1]⁺. Anal. (C₂₅H₄₃NO₄) C, H, N.

11-(2-Hexyl-5-hydroxy-phenoxy)-undecanoic acid cyclopropylamide (52). White solid (CHCl₃/MeOH=47/3) (90% yield): m.p. 57.3° C. (M). H-NMR 300 MHz (DMSO) δ (ppm): 9.05 (s, 1H), 7.76 (s br, 1H), 6.81 (d, 1H, J=7.7 Hz), 6.29 (s, 1H), 6.20 (d, 1H, J=8.1 Hz), 3.83 (t, 2H, J=5.5 Hz), 2.58-2.54 (m, 1H), 2.38 (t, 2H, J=7.3 Hz), 1.95 (t, 2H, J=6.9 Hz), 1.69-1.64 (m, 2H), 1.43-1.38 (m, 4H), 1.26-1.14 (mm, 18H), 0.82 (t, 3H, J=6.1 Hz), 0.57-0.53 (m, 2H), 0.33-0.31 (m, 2H). MS m/z: 418 [M+1]⁺, 440 [M+Na]⁺ (100). Anal. (C₂₆H₄₃NO₃) C, H, N.

11-(2-Hexyl-5-hydroxy-phenoxy)-undecanoic acid (4-hydroxy-phenyl)-amide (53). White solid (CHCl₃/MeOH=47/3; recrystallized diethyl ether) (83% yield): m.p. 108.7° C. (M). H-NMR (Acetone-d₆) δ (ppm): 9.05 (s, 1H), 9.00 (s, 1H), 8.80 (s, 1H), 7.43 (half of AB_{q}, 2H, J=8.8 Hz), 6.86 (d, 1H, J=7.9 Hz), 6.73 (half of AB_{q}, 2H, J=8.8 Hz), 6.40 (d, 1H, J=2.5 Hz), 6.30 (dd, 1H, J=2.5 Hz, J=7.9 Hz), 3.90 (t, 2H, J=6.1 Hz), 2.48 (t, 2H, J=7.4 Hz), 2.28 (t, 2H, J=7.2 Hz), 1.80-1.60 (mm, 14H), 1.50-1.32 (mm, 10H), 0.86 (t, 3H, J=6.3 Hz). Anal. (C₂₉H₄₃NO₄) C, H, N.

16-(2-Hexyl-5-hydroxy-phenoxy)-hexadecanoic acid (2-hydroxy-ethyl)-amide (54). White solid (Ethyl acetate/MeOH=50/1) (60% yield): m.p. 77.2° C. (M). H-NMR (DMSO) δ (ppm): 9.00 (s, 1H), 7.64 (s br, 1H), 6.80 (d, 1H, J=8.0 Hz), 6.28 (d, 1H, J=1.7 Hz), 6.20 (dd, 1H, J=8.0 Hz, J=1.7 Hz), 4.53 (t, 1H, J=5.2 Hz), 3.83 (t, 2H, J=5.8 Hz), 3.40-3.33 (m, 2H), 3.10-3.05 (m, 2H), 2.38 (t, 2H, J=7.3 Hz), 2.00 (t, 2H, J=7.4 Hz), 1.66-1.63 (m, 2H), 1.41-1.15 (mm, 32H), 0.82 (t, 3H, J=5.9 Hz). Anal. (C₃₀H₅₃NO₄) C, H, N.

16-(2-Hexyl-5-hydroxy-phenoxy)-hexadecanoic acid cyclopropylamide (55). White solid (CHCl₃/MeOH=47/3) (80% yield): m.p. 72.8° C. (M). H-NMR 300 MHz (DMSO) δ (ppm): 9.05 (s, 1H), 7.75 (s br, 1H), 6.81 (d, 1H, J=8.1 Hz), 6.29 (d, 1H, J=2.4 Hz), 6.20 (dd, 1H, J=2.4 Hz, J=8.1 Hz), 3.83 (t, 2H, J=6.3 Hz), 2.56-2.51 (m, 1H), 2.38 (t, 2H, J=7.2 Hz), 1.95 (t, 2H, J=7.2 Hz), 1.69-1.62 (m, 2H), 1.42-1.20 (mm, 32H), 0.82 (t, 3H, J=6.3 Hz), 0.56-0.52 (m, 2H), 0.34-0.31 (m, 2H). Anal. (C₃₁H₅₃NO₃) C, H, N.

16-(2-Hexyl-5-hydroxy-phenoxy)-hexadecanoic acid (4-hydroxy-phenyl)-amide (56). White solid (CHCl₃/MeOH=46/4) (70% yield): m.p. 82.4° C. (M). H-NMR (DMSO) δ (ppm): 9.45 (s, 1H), 9.05 (s, 1H), 9.02 (s, 1H), 7.30 (half of AB_{q}, 2H, J=8.7 Hz), 6.91 (d, 1H, J=8.0 Hz), 6.75 (half of AB_{q}, 2H, J=8.7 Hz), 6.35 (d, 1H, J=2.2 Hz), 6.29 (dd, 1H, J=2.2 Hz, J=8.0 Hz), 3.88 (t, 2H, J=6.3 Hz), 2.40 (t, 2H, J=7.5 Hz), 2.33 (t, 2H, J=7.5 Hz), 1.80-1.62 (m, 4H), 1.58-1.25 (mm, 30H), 0.86 (t, 3H, J=6.5 Hz). Anal. (C₃₄H₅₃NO₄) C, H, N.

### Pharmacological Methods

Procedure for assaying CB1 and CB2 binding

The synthesised compounds were assayed on recombinant human receptors CB₁ or CB2 over-expressed in COS cells, as described by the manufacturer (Perkin-Elmer). In short, increasing concentrations of the compounds to be assayed were incubated with 4-8 μg of membranes from transfected COS cells in the presence of 0.1-0.3 nM [³H]CP55,940 for 90 minutes at 30° C. in a buffer solution for binding in the absence of PMSF. After incubation, 0.1-0.3 nM [³H]CP55,940 bound and unbound was separated by filtration. The non specific binding was determined with 10 μM WIN 55-212, a high affinity ligand of CB₁ and CB₂ receptors, and it was never found to exceed 10%. IC₅₀ values in nM were obtained from dose-response curves using GraphPad® and transforming them into Ki using the Cheng-Prusoff equation.

Data are the mean ±SD of n=3 separate experiments and they are expressed as Ki in nM.

### Assay for Fatty Acid Amide Hydrolase (FAAH)

The effect of compounds on the enzymatic hydrolysis of [¹⁴C]anandamide (6 μM) was studied using membranes prepared from rat brain incubated with increasing concentrations of compounds in 50 mM Tris-HCl, pH 9, for 30 minutes at 37° C. (Di Marzo et al., 2002). [¹⁴C]ethanolamine produced from [¹⁴C]anandamide hydrolysis was measured counting the scintillation of the aqueous phase after extraction of the incubation mixture with 2 volumes of CHCl₃/CH₃OH (2:1 by volume). In most cases, only the more potent compounds in the binding assay to CB1 and CB2 were subjected to this analysis.

### Assay for the Anandamide Membrane Transporter (AMT)

The effect of the compounds on the uptake of anandamide by the RBL-2H3 cells was studied as described (Di Marzo et al., 2002). Cells were incubated with [¹⁴C]anandamide (4 μM) for 5 minutes at 37° C., in the presence or absence of varying concentrations of the inhibitors. Residual [¹⁴C]anandamide in the incubation media after extraction with CHCl₃/CH₃OH (2:1 by volume) was used as a measure of the anandamide that was taken up by cells. Data are expressed as the concentration that produces 50% of inhibition of anandamide uptake (IC50) calculated with GraphPad. In most cases, only the more potent compounds in the CB1 and CB2 binding assay were subjected to this analysis.

### Assay for Cyclic AMP

The assay for cyclic AMP was performed on CHO cells over-expressing recombinant CB 1 or CB2 receptors or on intact confluent N18TG2 cells plated in six-well plates and stimulated for 10 minutes at 37° C. with forskolin 1 μM in 400 μl of serum free DMEM which contains 20 mM HEPES, 0.1 mg/mL BSA, 0.1 mM 1-methyl-3-isobutylxanthine (Melck et al., 1999). Cells were treated with the solvent alone (methanol, 0.1%), or with the compounds, or with WIN55,212-2 at various concentrations, or with SR141716A (100 nM). After incubation, 800 μl of methanol were added, cells were extracted and cyclic AMP was determined with a cyclic AMP assay kit (Amersham, UK), in accordance with the supplier's recommendations.

### GTP-γ-S-binding

These assays were performed as previously described (Thomas et al., 2005), using mouse brain membranes containing CB1 receptors or CHO cells stably over-expressing the human recombinant CB₂ receptor (10 μg/ml protein), 0.7 nM [³⁵S]GTP-γ-S and 320 μM GDP, and a final assay volume of 250 μl. Results were compared to the potent CB₁ and CB₂ receptor agonist CP55940, purchased from Cayman Chemicals, Ann Arbour, Mich., USA.

### RESULTS

### Binding to CB1, CB2, FAAH and AMT

The results of affinity to CB1, CB2, FAAH and AMT are summarised in Table 1.

The authors' preliminary goal was to evaluate the hypothesis that joining the chemical stability of THC with the flexibility of AEA in a single structure could lead to compounds active on the endocannabinoid system.

For this purpose, the authors introduced an aliphatic chain that transports an amidic “head” in the aromatic structure of 3-pentadecylphenol, olivetol, resorcinol and 4-hexylresorcinol, realising five series of O-alkylate derivatives from which the authors obtained useful information on the structure-activity relationships. The binding results of the residues described in the present invention enable to establish the effect of many factors on the affinity for the CB1 and CB2 receptors and in the most important points can be summarised as follows:

(a) the presence of a hydroxy-phenolic group plays an essential role, since without it the derivatives of 3-pentadeciphenol 15-17 cannot bind to the cannabinoid receptors with high affinity;

(b) the length of the aliphatic chain on the aromatic ring has a crucial influence on the affinity of the residues, because a chain of five or six carbon atoms, as in olivetol and in 4-hexylresorcinol, is required, whilst a longer chain, as in 3-pentadecylphenol, or the absence of a chain, as in resorcinol, leads respectively to insoluble compounds (18-20) or inactive compounds (30-38);

(c) with regard to the alkyloxy chain that transports the amidic “head”, its length was also found to have great importance: short derivatives with five carbon atom chain (15-17, 21-23, 30-32, 39-41 and 48-50) or long derivatives with fifteen carbon atoms (27-29, 36-38, 45-47 and 54-56) are inactive regardless of their aromatic structure. In support of this analysis, it can be observed that the more potent compounds are olivetol derivatives (24, 25 and 26 ) and those of 4-hexylresorcinol (51 and 52), all with a chain of ten carbon atoms; a notable exception to this rule is the compound 40 which is more potent than 43 to bind to the receptor CB1 and equipotent to bind to the receptor CB2;

(d) consistently with literature data (Di Marzo et al., 1999), cyclopropylamides (25, 43 and 52) are more potent than the respective ethanolamides (24, 42 and 51) and in particular, special attention should be paid to the compound 25 (FIG. 1), which exhibits the lowest values of Ki and behaves like a very powerful ligand for CB1 (5.2 nM) and CB2 (13 nM), with affinity similar to WIN 55-212 (CB1 21±1.1 nM and CB2 2.1±0.1 nM);

(e) Moreover, it seems interesting to compare the two isomers of 4-hexylresorcinol: whilst the compound 52 is a powerful ligand of CB2 (30 nM), although it is not very selective with respect to the CB1 receptors (210 nM), the related regioisomer 43, albeit less potent, is a selective ligand for CB2 with at least 26 times selectivity with respect to the CB1 receptors (CB2 0.35 μM and CB1>10 μM);

(f) with regard to the interaction of the most potent ligands for the cannabinoid receptors with FAAH and AMT, the data obtained in the present invention show that all these compounds do not bind to the enzyme nor to the presumed transporter.

TABLE I

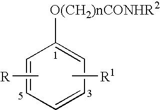

| Comp. | R | R₁ | n | R₂ | CB₁ | CB₂ | FAAH | AMT |
|---|---|---|---|---|---|---|---|---|
| 15 | H | 3-CH₂(CH₂)₁₃CH₃ | 5 | CH₂CH₂OH | n.t. | n.t. | n.a. | >25 |
| 16 | H | 3-CH₂(CH₂)₁₃CH₃ | 5 | (c•C₃H₅) | n.t. | n.t. | n.a. | n.t. |
| 17 | H | 3-CH₂(CH₂)₁₃CH₃ | 5 | p•OH—C₆H₄ | n.t. | n.t. | n.a. | >25 |
| 18 | H | 3-CH₂(CH₂)₁₃CH₃ | 10 | CH₂CH₂OH | n.t. | n.t. | n.t. | n.t. |
| 19 | H | 3-CH₂(CH₂)₁₃CH₃ | 10 | c•C₃H₅ | n.t. | n.t. | n.t. | n.t. |
| 20 | H | 3-CH₂(CH₂)₁₃CH₃ | 10 | p•OH—C₆H₄ | n.t. | n.t. | n.t. | n.t. |
| 21 | 3-OH | 5-CH₂(CH₂)₃CH₃ | 5 | CH₂CH₂OH | n.t. | n.a. | n.a. | n.t. |
| 22 | 3-OH | 5-CH₂(CH₂)₃CH₃ | 5 | c•C₃H₅ | 1.3 | 0.96 | n.a. | n.t. |
| 23 | 3-OH | 5-CH₂(CH₂)₃CH₃ | 5 | p•OH—C₆H₄ | n.t. | n.t. | n.a. | n.t. |
| 24 | 3-OH | 5-CH₂(CH₂)₃CH₃ | 10 | CH₂CH₂OH | 0.8 | 0.16 | n.a. | 25. |
| 25 | 3-OH | 5-CH₂(CH₂)₃CH₃ | 10 | c•C₃H₅ | 0.0052 | 0.013 | n.a. | 17 |
| 26 | 3-OH | 5-CH₂(CH₂)₃CH₃ | 10 | p•OH—C₆H₄ | 3 | 1.4 | n.a. | n.a. |
| 27 | 3-OH | 5-CH₂(CH₂)₃CH₃ | 15 | CH₂CH₂OH | 12.5 | n.a. | n.t. | n.t. |
| 28 | 3-OH | 5-CH₂(CH₂)₃CH₃ | 15 | c•C₃H₅ | >10 | n.a. | n.t. | n.t. |
| 29 | 3-OH | 5-CH₂(CH₂)₃CH₃ | 15 | p•OH—C₆H₄ | >10 | n.a. | n.t. | n.t. |
| 30 | 3-OH | H | 5 | CH₂CH₂OH | n.t. | n.t. | n.a. | n.t. |
| 31 | 3-OH | H | 5 | c•C₃H₅ | n.t. | n.t. | n.a. | n.t. |
| 32 | 3-OH | H | 5 | p•OH—C₆H₄ | n.t. | n.t. | n.t. | n.t. |
| 33 | 3-OH | H | 10 | CH₂CH₂OH | >10 | n.a. | n.a. | n.t. |
| 34 | 3-OH | H | 10 | c•C₃H₅ | >10 | 5.4 | n.a. | n.t. |
| 35 | 3-OH | H | 10 | p•OH—C₆H₄ | n.a. | >10 | n.t. | n.t. |
| 36 | 3-OH | H | 15 | CH₂CH₂OH | >10 | n.a. | n.t. | n.t. |
| 37 | 3-OH | H | 15 | c•C₃H₅ | 4.25 | n.a. | n.t. | n.t. |
| 38 | 3-OH | H | 15 | p•OH—C₆H₄ | 5 | 10 | n.t. | n.t. |
| 39 | 3-OH | 4-CH₂(CH₂)₄CH₃ | 5 | CH₂CH₂OH | n.t. | 6.43 | n.a. | n.t. |
| 40 | 3-OH | 4-CH₂(CH₂)₄CH₃ | 5 | c•C₃H₅ | 0.18 | 0.54 | n.a. | n.t. |
| 41 | 3-OH | 4-CH₂(CH₂)₄CH₃ | 5 | p•OH—C₆H₄ | n.t. | n.t. | n.t. | n.t. |
| 42 | 3-OH | 4-CH₂(CH₂)₄CH₃ | 10 | CH₂CH₂OH | >10 | 2.7 | n.a. | n.a. |
| 43 | 3-OH | 4-CH₂(CH₂)₄CH₃ | 10 | c•C₃H₅ | >10 | 0.35 | n.a. | >25 |
| 44 | 3-OH | 4-CH₂(CH₂)₄CH₃ | 10 | p•OH—C₆H₄ | n.a. | >10 | 50 | >25 |
| 45 | 3-OH | 4-CH₂(CH₂)₄CH₃ | 15 | CH₂CH₂OH | n.a. | n.a. | n.t. | n.t. |
| 46 | 3-OH | 4-CH₂(CH₂)₄CH₃ | 15 | c•C₃H₅ | n.a. | n.a. | n.t. | n.t. |
| 47 | 3-OH | 4-CH₂(CH₂)₄CH₃ | 15 | p•OH—C₆H₄ | >10 | n.a. | n.t. | n.t. |
| 48 | 5-OH | 2-CH₂(CH₂)₄CH₃ | 5 | CH₂CH₂OH | n.t. | >10 | n.a. | n.t. |
| 49 | 5-OH | 2-CH₂(CH₂)₄CH₃ | 5 | c•C₃H₅ | n.t | 3.57 | n.a. | n.t. |
| 50 | 5-OH | 2-CH₂(CH₂)₄CH₃ | 5 | p•OH—C₆H₄ | n.t. | n.t. | n.t. | n.t. |
| 51 | 5-OH | 2-CH₂(CH₂)₄CH₃ | 10 | CH₂CH₂OH | 1.13 | 0.42 | n.a. | >25 |
| 52 | 5-OH | 2-CH₂(CH₂)₄CH₃ | 10 | c•C₃H₅ | 0.21 | 0.03 | >25 | 13 |
| 53 | 5-OH | 2-CH₂(CH₂)₄CH₃ | 10 | p•OH—C₆H₄ | 3.3 | 2.11 | 18.0 | >25 |
| 54 | 5-OH | 2-CH₂(CH₂)₄CH₃ | 15 | CH₂CH₂OH | n.a. | n.a. | n.t. | n.t. |
| 55 | 5-OH | 2-CH₂(CH₂)₄CH₃ | 15 | c•C₃H₅ | 5.5 | 2.49 | n.t. | n.t. |
| 56 | 5-OH | 2-CH₂(CH₂)₄CH₃ | 15 | p•OH—C₆H₄ | 3 | n.a. | n.t. | n.t. |

| | | |
|---|---|---|
| anandamide | 72 ± 3.1 | — |
| | nM | |
| WIN, 55-212 | 21 ± 1.1 | 2.1 ± 0.1 |
| | nM | nM |
| HU-210 | — | 0.15 ± 0.0 |
| | | 3 |

The data are mean ± SEM of n = 3 separate examples and they are expressed as Ki (μM), for the bond to CB1 and CB2 and in IC50 (μM) for the bond to AMT and FAAH. The reference compounds were examined in the samde conditions. Anandamide was examined in the presence of PMSF (100 mM). n.a. = IC50 >10 in the preliminary selection conducted with rat brain and spleen membranes; n.t. = not tested; insol. = insoluble in any of the solvents normally used in bond studies (DMSO,ethanol or methanol). Affinity constants of the residues: the more potent (Ki <μM) are highlighted with bold characters.

### Activity on Cyclic AMP and GTP-γ-S-binding

To understand whether the ligands described herein are able functionally to modify the activity of the cannabinoid receptors, i.e. to behave as agonists or antagonists, the functional activity at cannabinoid receptors of the two compounds with the highest affinity for CB₁ and/or CB₂ receptors, i.e. compounds 25 and 52 was tested. Their capability to inhibit forskolin-induced adenylyl cyclase assay in intact cells (Test 1) or to stimulate GTP-γ-S-binding to cell membranes (Test 2) was evaluated.

Compound 25, was tested for its activity on the induced adenylate cyclase of forskolin in N18TG2 mouse neuroblastoma cells, which constitutively and selectively express the CB 1 cannabinoid receptors. As indicated in FIG. 2, similarly to the reference inverse agonist/antagonist for the CB1 receptors, SR141716A, the compound 25 significantly stimulated the induced formation of AMPc of forskolin (half-maximum effect observed at a concentration of 13 nM similar to the Ki for this compound to CB1 receptors). As expected, WIN55,212-2 behaved as an agonist, inhibiting the induced formation of AMPc of forskolin (half-maximum effect observed at a concentration of 15 nM). The effect of the compound 25 was mediated by the CB1 receptors because it was blocked with a dose that itself was inactive (5 nM) of WIN55,212 (FIG. 2). Consequently 25 behaves as an inverse agonist/antagonist of CB1 receptors. Moreover, another compound that exhibited high affinity for CB1 receptors, albeit lower than the compound 25, i.e. the compound 40, also behaved as inverse agonist/antagonist (half-maximum effect observed at a concentration of 150 nM).

When Test 1 was carried out in CHO cells over-expressing the human recombinant CB₁ receptor, the compounds 25 and 52 behave as partial agonists (Table II). Indeed, both compounds inhibit, rather than stimulate, forskolin-induced cAMP formation.

TABLE II
Effect of compounds 25 and 52 on forskolin-induced
cAMP formation in CHO cells over-expressing the human
recombinant CB₁ and CB₂ receptors.

| | hCB₁ | | hCB₂ | |
|---|---|---|---|---|
| | % effect with 10 μM | EC₅₀ | % effect with 10 μM | EC₅₀ |
| CB 25 | 63% | 1.6 μM | 26% | >10 μM |
| CB 52 | 62% | 2.4 μM | 5% | >10 μM |

This latter finding was confirmed using Test 2 in membranes from mouse cells (FIG. 3). In this case, in fact, compounds 25 (IC₅₀=0.15 μM) and 52 (IC₅₀=0.10 μM) stimulated GTP-γ-S-binding to mouse brain membranes, which indicates an agonist action.

However, when compared to the potent CB₁ and CB₂ agonist CP55940, compound 52 was less efficacious than what observed in Test 1. Furthermore, both compounds were more potent in Test 2 than in Test 1.

Regarding the functional activity of the two compounds at CB₂ receptors, both Test 1 and Test 2, carried out in CHO cells over-expressing the human recombinant CB₂ receptor, showed that both 25 and 52 behave as neutral (“silent”) antagonists of this receptor (Table II, FIG. 4, 5, 6). Indeed, the two compounds exerted no significant effect on forskolin-induced cAMP formation and no significant stimulation or inhibition of GTP-γ-S-binding to mouse brain membranes. In addition, both compounds block the effect of CP55940 in this assay.

Therefore, the compounds 25 and 52 are the first compounds ever synthesised to have overall activity as CB1 receptor agonists and, at the same time, “silent” antagonistic activity at the CB2 receptor.

These findings indicate that the two compounds couple the beneficial actions usually associated with CB₁ receptor agonists (anti-spastic activity in multiple sclerosis, pain relief, anti-cancer activity, etc.) with those that are being found for CB₂ receptor antagonists (anti-inflammatory activities in various assays in vitro and in vivo).

### BIBLIOGRAPHY

Calignano A. et al., European Journal of Pharmacology, 337, 1, 1997, R1-R2.

Devane W. A. et al., Mol. Pharmacol., 1988, 34, 605-613.

Devane, W. A. et al., Science 1992, 258, 1946-1949.

Di Marzo V et al., Current Medicinal Chemistry, 6, 1999, 721.

Di Marzo V. et al., J. Pharmacol. Exp. Ther., 300, 2002, 984-991.

Di Marzo, V. et al., Nat. Rev. Drug Disc., 2004, 3, 771-784.

Goutopoulos A. et al., Pharmacology Therapeutics, 95(2), 103-107, 2002.

Hansen H. S. et al., Biochem. Pharmacol., 55, 1998, 719-725.

Mechoulam, R. et al., Biochem. PharmacoL, 1995, 50, 83-90.

Melck, D. et al., FEBS Lett., 463(3), 1999, 235-40.

Munro, S. et al., Nature 1993, 365, 61-65.

Thomas, A., et al. Br. J. Pharmacol. 2005, 146, 917-26.

Varga et al., Faseb. J, 12, 1998, 1035-1044.

Wagner et al., Nature, 390, 1997, 518-521.

1. Compound with the formula 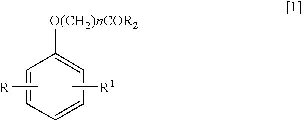
where
n is a number from 4-14 [1-15];
R represents [a hydrogen atom, a halogen,] a hydroxyl group [or an alkyloxy group];
R, represents [a hydrogen atom, a halogen (chlorine, bromine, iodine, fluorine), a hydroxyl group, or] an alkyloxy group or a saturated or unsaturated alkylic chain of various lengths;
R2 represents a secondary amine (ethanolamine, propanolamine, propylamine, isopropylamine, cyclopropylamine, cyclopropylmethylamine, 2-alogen-ethylamines).

2. (canceled)

3. (canceled)

4. (canceled)

5. (canceled)

6. (canceled)

7. (canceled)

8. Compound as claimed in claim 1 being the 6-(3-hydroxy-5-pentil-phenoxy)-hexanoic acid (2-hydroxy-ethyl)-amide.

9. Compound as claimed in claim 1 being the 6-(3-Hydroxy-5-pentil-phenoxy)-hexanoic acid cyclopropylamide.

10. Compound as claimed in claim 1 being the 6-(3-hydroxy-5-pentil-phenoxy)-hexanoic acid (4-hydroxy-phenyl)amide.

11. Compound as claimed in claim 1 being the 11-(3-Hydroxy-5-pentil-phenoxy)-undecanoic acid (2-hydroxy-ethyl)-amide.

12. Compound as claimed in claim 1 being the 11-(3-hydroxy-5-pentilphenoxy) undecanoic acid cyclopropylamide.

13. Compound as claimed in claim 1 being the 11-(3-liydroxy-5-pentil-phenoxy)-undecanoic acid (4-hydroxy-phenyl)-amide.

14. (canceled)

15. (canceled)

16. (canceled)

17. Compound as claimed in claim 1 being the 6-(3-hydroxy-phenoxy)-hexanoic acid (2-hydroxy-ethyl)-amide.

18. Compound as claimed in claim 1 being the 6-(3-hydroxy-phenoxy)-hexanoic acid cyclopropylamide.

19. Compound as claimed in claim 1 being the 6-(3-hydroxy-phenoxy)-hexanoic acid (4-hydroxy-phenyl)-amide.

20. Compound as claimed in claim 1 being the 11-(3-hydroxy-phenoxy)-undecanoic acid (2-hydroxy-ethyl)-amide.

21. Compound as claimed in claim 1 being the 11-(3-hydroxy-phenoxy)-undecanoic acid cyclopropylamide.

22. Compound as claimed in claim 1 being the 11-(3-hydroxy-phenoxy)-undecanoic acid (4-hydroxy-phenyl)-amide.

23. (canceled)

24. (canceled)

25. (canceled)

26. Compound as claimed in claim 1 being the 6-(3-hydroxy-4-hexyl-phenoxy)-hexanoic acid (2-hydroxy-ethyl)-amide.

27. Compound as claimed in claim 1 being the 6-(3-hydroxy-4-hexyl-phenoxy)-hexanoic acid cyclopropylamide.

28. Compound as claimed in claim 1 being the 6-(3-hydroxy-4-hexyl-phenoxy)-hexanoic acid (4-hydroxy-phenyl)-amide.

29. Compound as claimed in claim 1 being the 11-(3-hydroxy-4-hexyl-phenoxy)-undecanoic acid (2-hydroxy-ethyl)-amide.

30. Compound as claimed in claim 1 being the 11-(3-hydroxy-4-hexyl-phenoxy)-undecanoic acid cyclopropylamide.

31. Compound as claimed in claim 1 being the 11-(3-hydroxy-4-hexyl-phenoxy)-undecanoic acid (4-hydroxy-phenyl)-amide.

32. (canceled)

33. (canceled)

34. (canceled)

35. (canceled)

36. Compound as claimed in claim 1 being the 6-(2-hexyl-5-hydroxy-phenoxy)-hexanoic acid cyclopropylamide.

37. Compound as claimed in claim 1 being the 6-(2-hexyl-5-hydroxy-phenoxy)-hexanoic acid (4-hydroxy-phenyl)-amide.

38. Compound as claimed in claim 1 being the 11-(2-hexyl-5-hydroxy-phenoxy)-undecanoic acid (2-hydroxy-ethyl)-amide.

39. Compound as claimed in claim 1 being the acid 11-(2-hexyl-5-hydroxy-phenoxy)-undecanoic cyclopropylamide.

40. Compound as claimed in claim 1 being the 11-(2-hexyl-5-hydroxy-phenoxy)-undecanoic acid (4-hydroxy-phenyl)-amide.

41. Compound as claimed in claim 1 being the 16-(2-hexyl-5-hydroxy-phenoxy)-hexadecanoic acid (2-hydroxy-ethyl)-amide.

42. Compound as claimed in claim 1 being the 16-(2-hexyl-5-hydroxy-phenoxy)-hexadecanoic acid cyclopropylamide.

43. Compound as claimed in claim 1 being the 16-(2-hexyl-5-hydroxy-phenoxy)-hexadecanoic acid (4-hydroxy-phenyl)-amide.

44. Compound as claimed in claim 1, able to bind at least one of the receptors of the endocannabinoid system.

45. A pharmaceutical composition, comprising: a pharmaceutically effective amount of a compound of claim 1 for medical use, and a pharmaceutically acceptable carrier.

46. A method of treating a patient in need thereof, comprising: administering to said patient an effective amount of the pharmaceutical composition of claim 45, wherein the patient has a condition which requires treatment with an agent for pain therapy, inflammation, stress, oxidative therapy, hypotensive therapy, immunosuppressive therapy, obesity therapy, metabolic syndrome therapy, spastic activity in multiple sclerosis and/or anti-cancer.
